(19) United States
(12) Patent Application Publication
(10) Pub. No.: US 2024/0050697 A1
(43) Pub. Date: Feb. 15, 2024

(54) CANNULATION SYSTEMS AND METHODS

(71) Applicant: Vitara Biomedical, Inc., Philadelphia, PA (US)

(72) Inventors: Christopher C. GREGORY, Newtown, PA (US); Jeff KIERSH, Bethlehem, PA (US)

(21) Appl. No.: 18/266,431

(22) PCT Filed: Dec. 10, 2021
(86) PCT No.: PCT/US2021/062867

§ 371 Date: Jun. 9, 2023

Related U.S. Application Data
(60) Provisional application No. 63/124,314, filed on Dec. 11, 2020.

(51) Int. Cl. A61M 25/00; A61M 39/22

(52) U.S. Cl. CPC A61M 25/0097; A61M 39/223; A61M 39/221; A61M 2039/222; A61M 2039/229

(57) ABSTRACT

One aspect of the invention provides a system including: a cannula and a valve. The valve includes: a first port coupled to the cannula; a second port adapted and configured for coupling to tubing; a third port comprising a resilient pierceable seal; and a central cylindrical plug received between the first port, the second port, and the third port. The central plug includes a through-channel adapted and configured to selectively couple two of the ports. In a first position, the first port, the third port, and the through-channel are coupled such that a dilator can pass: from outside the rotary valve; through the pierceable seal; through the first port; and out a distal end of the cannula. In a second position, the first port and the second are port are fluidically coupled.

### CROSS-REFERENCE TO RELATED APPLICATION

This application claims the benefit of priority of U.S. Provisional Patent Application Ser. No. 63/124,314, filed Dec. 11, 2020. The entire content of this application is hereby incorporated by reference herein.

### BACKGROUND OF THE INVENTION

Cannulation of vessels such as blood vessels is required in order to access and return fluid from a subject.

### SUMMARY OF THE INVENTION

One aspect of the invention provides a system including: a cannula and a valve. The valve includes: a first port coupled to the cannula; a second port adapted and configured for coupling to tubing; a third port comprising a resilient pierceable seal; and a central cylindrical plug received between the first port, the second port, and the third port. The central plug includes a through-channel adapted and configured to selectively couple two of the ports. In a first position, the first port, the third port, and the through-channel are coupled such that a dilator can pass: from outside the rotary valve; through the pierceable seal; through the first port; and out a distal end of the cannula. In a second position, the first port and the second are port are fluidically coupled.

This aspect of the invention can have a variety of embodiments. The valve can be a rotary valve. The second port and the third port can be rotated relative to the first port. The central cylindrical plug can be fixed relative to the first port. The central cylindrical plug can be fixed relative to the second port and the third port. In one embodiment, the first port and the third port can be axially aligned. In an alternative embodiment, the first port and the third port can be diametrically opposed.

In one embodiment, the dilator can include a tapered distal end and a length relative to the cannula and the valve such that when the dilator is distally advanced through the rotary valve, the tapered distal end extends beyond a distal end of the cannula. The dilator can be adapted and configured to facilitate puncturing of a blood vessel.

The system can further include one or more centrally biased clamps adapted and configured to: spread radially during puncture of a blood vessel; and clamp the blood vessel against the cannula after puncture.

The cannula can have an outer diameter that ranges from 7 French to about 16 French.

In a third position, the through-channel can be in fluidic communication with one or none of the ports.

The valve can be a piston valve.

Another aspect of the invention provides a method of cannulating a vessel wherein fluid flows therethrough. The method includes: providing a valve including a first port, second port, and third port wherein a first port is coupled to a cannula and a second port is coupled to a length of tubing having a through channel; introducing a dilator distally through the third port of the valve; advancing the dilator and the cannula into the vessel; withdrawing the dilator from the cannula and the valve; and placing the valve in the second position to have the fluid flow between the vessel and the tubing.

This aspect of the invention can include a variety of embodiments. The vessel can be a blood vessel. The blood vessel can be an umbilical blood vessel. The method can be performed by a single person.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A-1D provides several views of a cannulation system according to an embodiment of the invention. FIG. 1A is a cross-sectional view of the system in which the rotary valve is in a first position prior to insertion of a dilator. FIG. 1B is a cross-sectional view of the system in which the rotary valve is in the first position after insertion of the dilator. FIG. 1C is side view of the system in which the rotary valve is in a second position after removal of the dilator. FIG. 1D is a cross-sectional view of the system in which the rotary valve is in the second position after removal of a dilator.

FIGS. 2A-2C provide perspective views of rotary valves according to an embodiment of the invention in which the first port and the central plug can be rotated relative to the second port and the third port. In FIG. 2A, all components are opaque, while central plug is partially transparent in FIGS. 2B and 2C.

FIGS. 3A and 3B provide a cross-sectional view of a rotary valve including a central plug with a through-channel having a flared end adjacent to the first port.

FIG. 4 depicts a method of cannulating a vessel according to an embodiment of the invention.

FIGS. 5A-5E provide perspective views of a cannulation system utilizing a trumpet or piston valve according to an embodiment of the invention. FIGS. 5B, 5C, and 5E are cross-sectional views to show selective coupling of ports.

FIGS. 6A and 6B provide cross-sectional views of a rotary valve having two through-channels in a common plane with the ports.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

The terms “proximal” and “distal” can refer to the position of a portion of a device relative to the remainder of the device or the opposing end as it appears in the drawing. The proximal end can be used to refer to the end manipulated by the user. The distal end can be used to refer to the end of the device that is inserted and advanced and is furthest away from the user. As will be appreciated by those skilled in the art, the use of proximal and distal could change in another context, e.g., the anatomical context in which proximal and distal use the patient as reference, or where the entry point is distal from the user.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

### Cannulation Systems

### Systems Including Rotary Valve

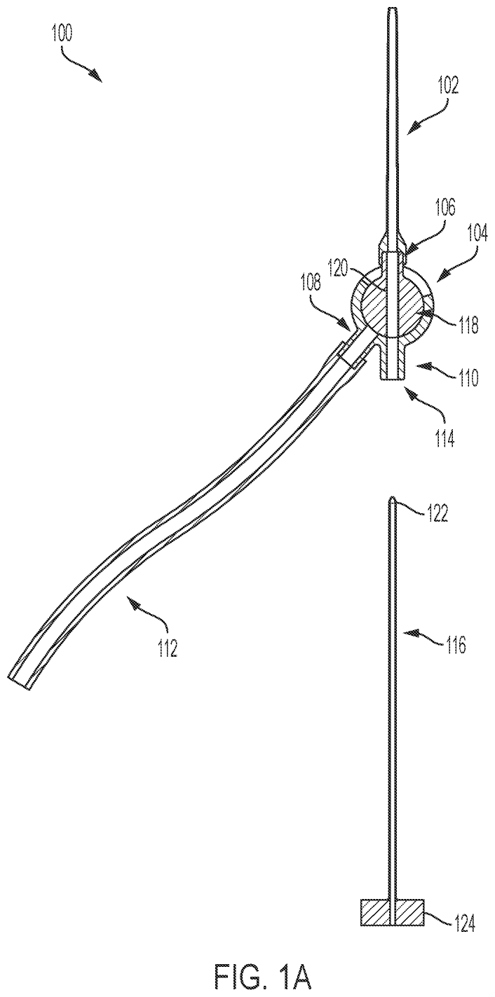

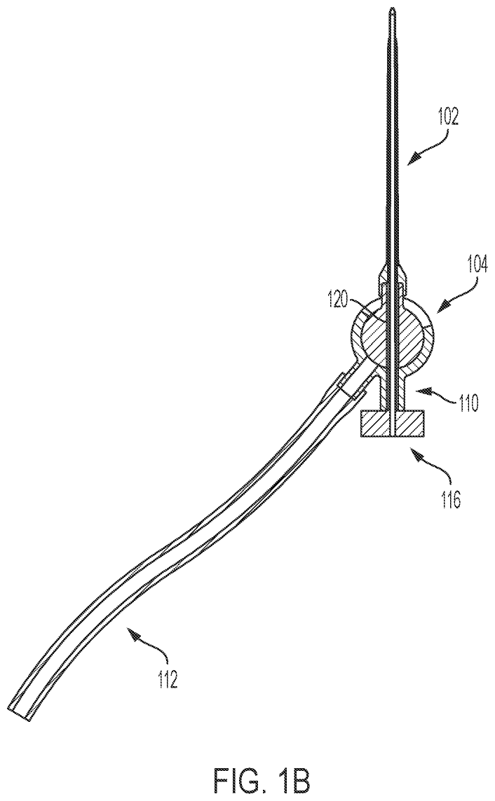

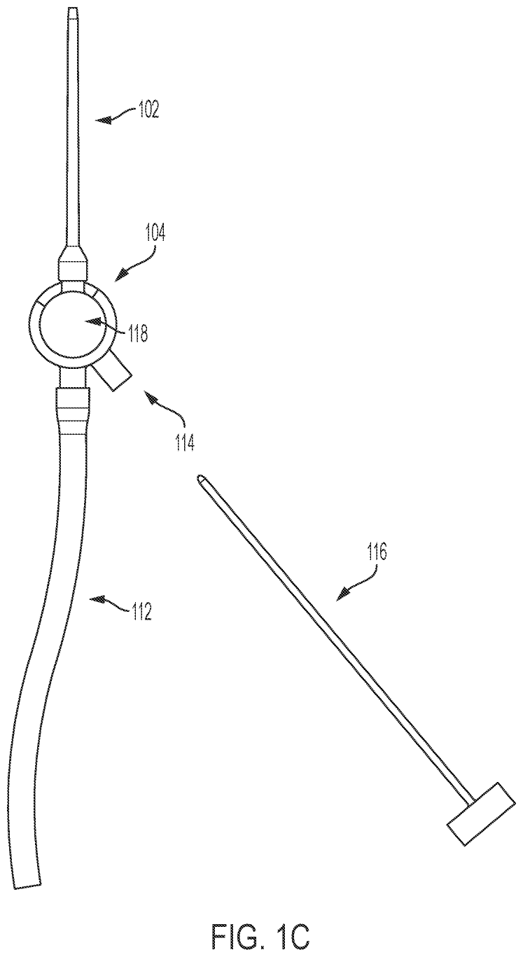

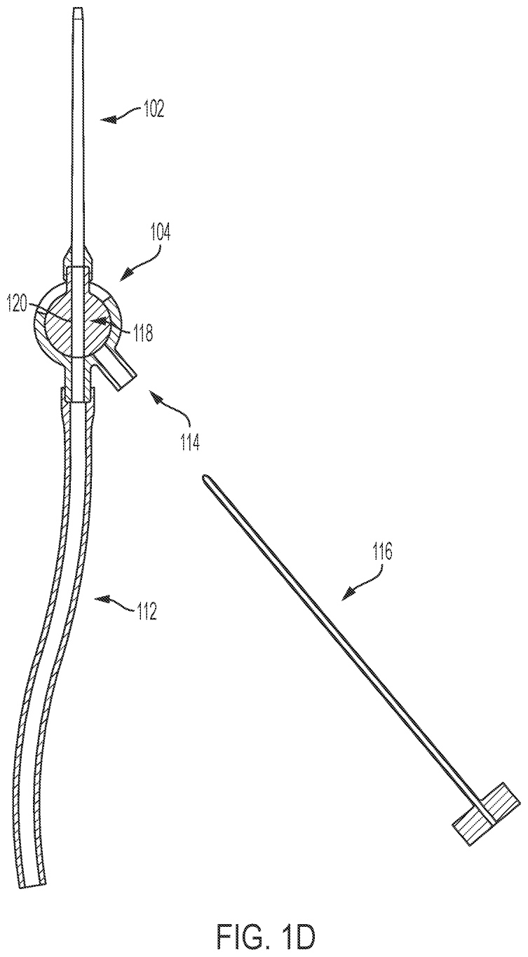

Referring to FIG. 1A, one aspect of the invention provides a system 100 including a cannula 102 and a valve 104 (a rotary valve in FIGS. 1A-1D). The cannula 102 can be coupled to the valve 104 as further described herein.

The cannula 102 can have a profile and length suitable for cannulating a vessel, e.g., a blood vessel. For example, the cannula 102 can have an outer diameter that ranges from about 7 French to about 16 French (e.g., from about 7 French to about 9 French, from about 14 French to about 16 French, and any range end-points in between).

The valve 104 can include a first port 106, a second port 108, and a third port 110.

The first port 106 can be coupled to the cannula 102.

The second port 108 can be adapted and configured for coupling to tubing 112.

The third port 110 (and/or other ports 106, 108) can include a resilient pierceable seal 114 such as a septum, duckbill, and the like. The resilient pierceable seal 114 can be an elastomer and can include one or more pre-defined passages. In one embodiment, seal 114 is puncturable by dilator 116. In another embodiment, the third port 110 can include a removable cap or seal. In any of the foregoing or in an alternative embodiment, the third port 110 remains fluid-tight up to 200 mmHg of pressure.

The rotary valve 102 can also include a central plug 118 received between the ports 106, 108, or 110. The central plug 118 can include a through-channel 120 adapted and configured to selectively couple any one of ports 108, 110 to port 106 when oriented appropriately. The rotary valve 104 can be moved (e.g., by rotation) between at least two positions. As depicted in FIG. 1A, through-channel 120 can have a substantially cylindrical profile that facilitates passage of dilator 116 from third port 110 across central plug 118 through first port 106 and into cannula 102 as depicted in FIG. 1B when in a first position as depicted in FIGS. 1A and 1B. Although the cylindrical through-channel 120 described herein advantageously provides a smooth blood path that avoids stagnation, eddies, or turbulence, other geometries can be used. For example, through-channel 120 need not be cylindrical, e.g., if dilator 116 is flexible and can follow a curved through-channel 120.

Dilator 116 can include a tapered (e.g., beveled, sharpened, and the like) distal end 122 capable of piercing tissue (e.g., the wall of a vessel such a blood vessel). For example, the dilator 116 can expand proximally from a distal point to a cross-sectional profile that approximates a distal cross-sectional dimension of the cannula 102. Dilator 116 can also include a proximal handle 124 to facilitate advancement and withdrawal of the dilator 116.

In one embodiment, the tolerance between dilator 116 and through-channel 120 and cannula 102 is tight to minimize dead volume that would be occupied by air that could be introduced into the vessel.

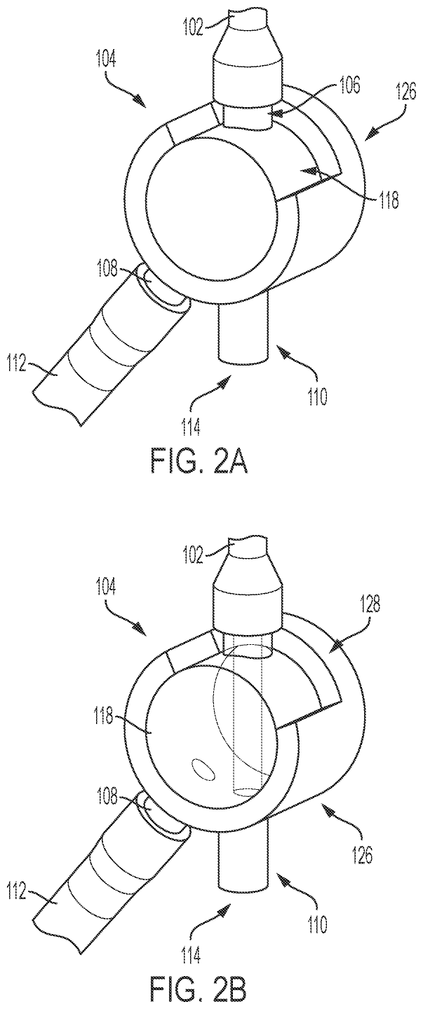

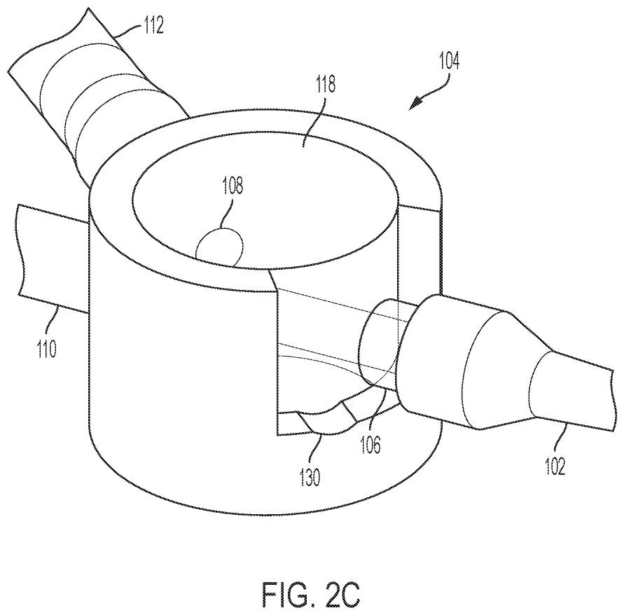

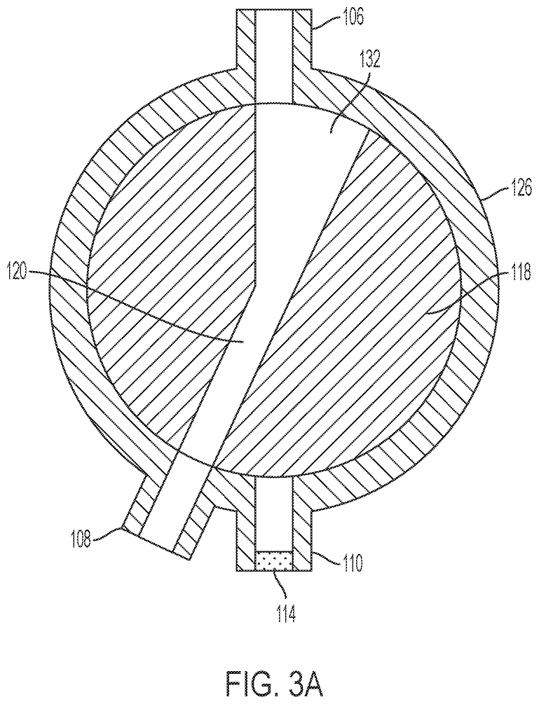

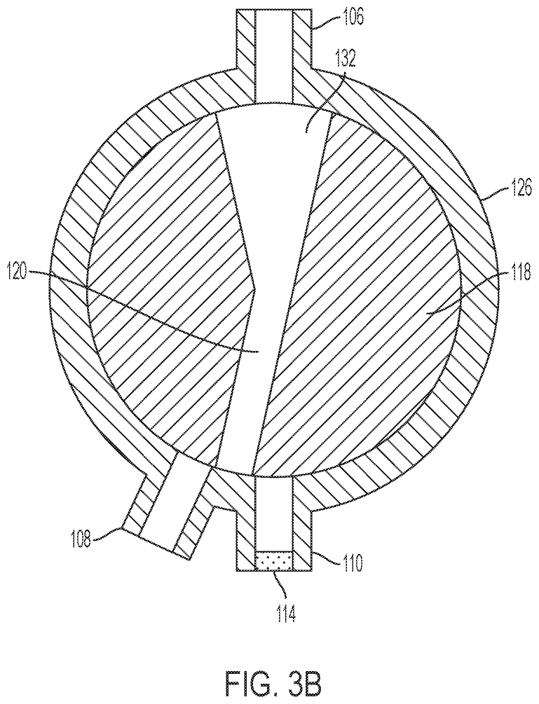

Referring now to FIG. 1C, after withdrawal of the dilator 116, the rotary valve can be actuated to a second position in which the first port 106 and second port 108 are fluidically coupled. This can be achieved in several ways. In one embodiment depicted in FIGS. 1A-2C the first port 106 and the central plug 118 can be coupled or integral such that the first port 106 and the central plug 118 can be rotated together, e.g., within a housing 126 defining second port 108 and third port 110 as depicted in FIGS. 1A-2B. Rotation and alignment with respect to the second port 108 or the third port 110 can be facilitated by a recess 128 in the housing 126 or a detent 130 in which first port 106 or another feature can selectively rest as depicted in FIG. 2C. In another embodiment, the second port 108 and the third port 110 can rotate relative to the central plug 118 and the first port 106. In still another embodiment, the through-channel 120 can include a flared end 132 adjacent to the first port 106 as depicted in FIGS. 3A and 3B.

Referring to FIG. 3B, in some embodiments, the valve 104 can be actuated to a third position (e.g., with through-channel 120 between second port 108 and third port 110 or between 106 and 110 or between 106 and 108) in which one or none of ports 106, 108, 110 are coupled. This would isolate cannula 102 if blood flow needed to be stopped.

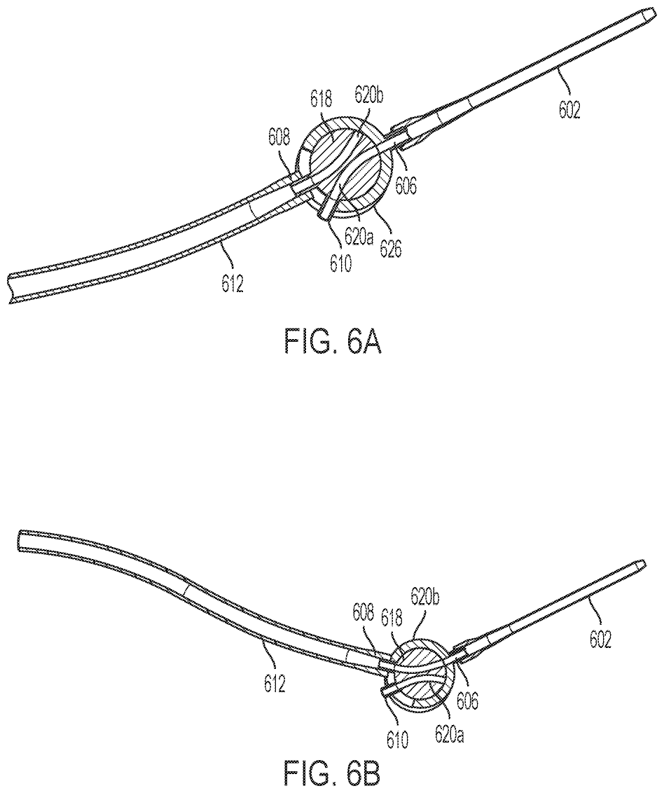

In some embodiments, the cannula can be connected to a port integrated with the valve housing and the second and third ports are integrated with the valve plug. Referring to the section view in FIG. 6A, the cannula 602 is connected to the first port 606 that is integrated with the housing 626. In a first position depicted in FIG. 6A, a channel in plug 618 creates a path from the second port 610 to the first port 606 allowing the introduction of a dilator (not shown), which may be flexible. In a second position, cross-sectionally depicted in FIG. 6B, the plug 618 is rotated relative to the housing 626 and a second channel creates a fluid path from the third port 608 to the first port 606.

While the embodiments shown herein refer to a first port, a second port, or a third port, it is envisioned that the valve can comprise a plurality of ports depending upon the nature of the vessel(s) to which the cannula will be fluidically coupled.

### Other Valves

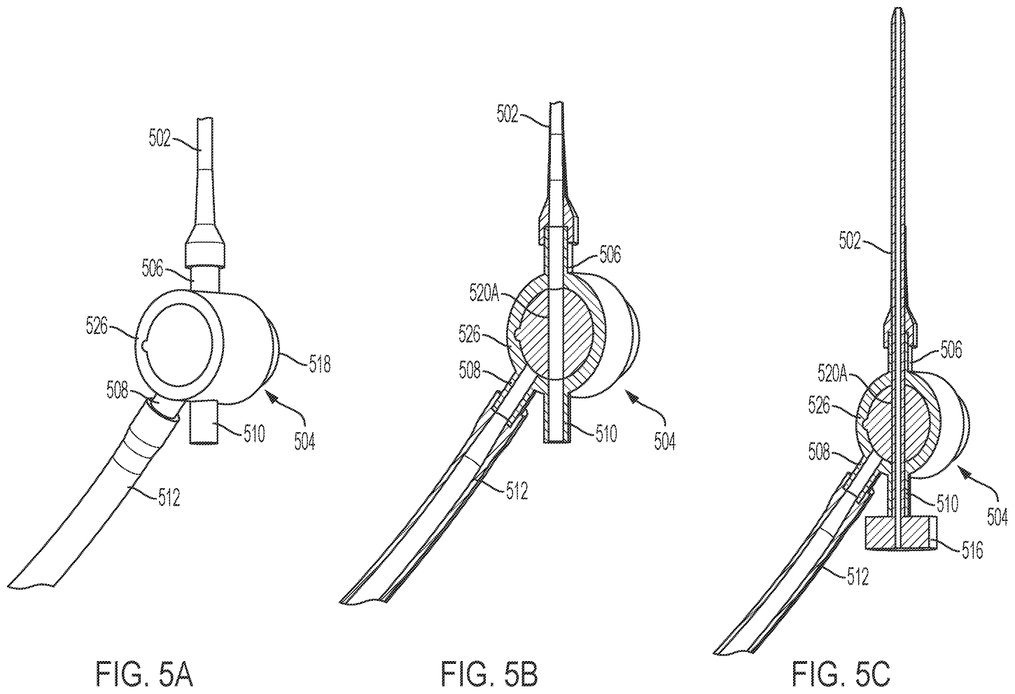

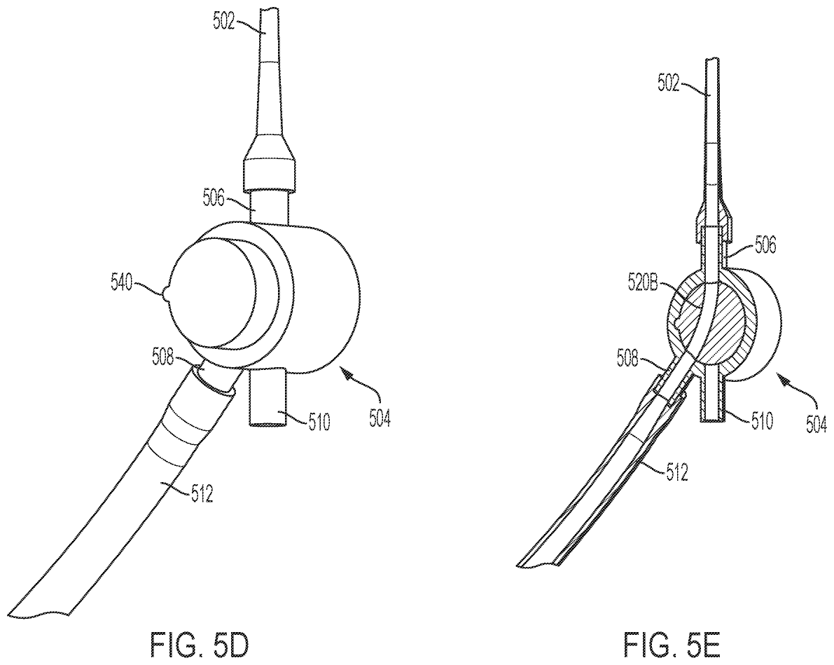

Referring now to FIGS. 5A-5E, embodiments of the invention can utilize other valves such as a trumpet, Périnet, or piston valve 504 or further including a piston 518 that can slide laterally within housing 526. The piston 518 contains two channels 520A and 520B offset from each other axially. In a first position, channel 520A connects the port 510 with port 506, creating a path from port 510 through to the cannula 502 (as depicted cross-sectionally in FIG. 5B) for a dilator 516 (as depicted cross-sectionally in FIG. 5C). In a second position, piston 518 is moved axially to the position shown in FIG. 5D aligning channel 530B with port 508 and port 506 creating a fluid-flow channel from the cannula 502 to fluid line 512. The piston 518 may have a non-circular profile or have an alignment feature such as 540 that corresponds to a complementary feature in the housing 526 to maintain rotational alignment between the piston 518 and the housing 526. Such a piston 518 could be moved with a thumb of the user's holding hand or could be spring-loaded to move to the second position (depicted in FIGS. 5D and 5E) after removal of a dilator 516.

### Clamping

Embodiments of the invention can incorporate one or more clamps to hold the cannula 102 in place within a vessel. For example, a variety of clamping devices such as centrally biased radial jaws are described in U.S. Provisional Patent Application Ser. No. 63/017,204, filed Apr. 29, 2020 and U.S. Patent Application Publication No. US 2021/0338270.

### Couplings

The various elements described herein (e.g., cannula 102 and valve 104, valve 104 and tubing 110, and the like) can be coupled during manufacturing, in the field, or at another time using a variety of techniques including mechanical elements (e.g., threads), cold working (e.g., crimping, swaging, and the like), interference fits, adhesives, and the like.

### Materials

The components described herein can be fabricated from a variety of biocompatible materials such as polymers, glass, metal, ceramics, and the like. Exemplary polymers include thermoplastic polymers, thermoset polymers, polycarbonate, polystyrene, PEEK, PES, ABS, polypropylene, polymethylpentene, polycarbonate, acetal products, polytetrafluoroethylene (PTFE) (e.g., TEFLON® available from The Chemours Company of Wilmington, Delaware), polysulfone products, DELRIN® acetal resin available from E. I. du Pont de Nemours and Company of Wilmington, Delaware, and the like.

One or more components can include an anti- or non-thrombogenic coating or additive.

### Methods of Manufacture

Components of the systems described herein can be fabricated using a variety of techniques including casting, molding, machining, thermomolding, thermosetting, injection molding, vacuum forming, additive manufacturing (also known as 3D printing), and the like.

In embodiments where the cannula port and the dilator port are aligned when in the first position, the through-channel 120 can be machined or drilled after assembly to ensure alignment of through-channel 120, first port 106, and third port 110.

### Methods of Cannulating a Vessel

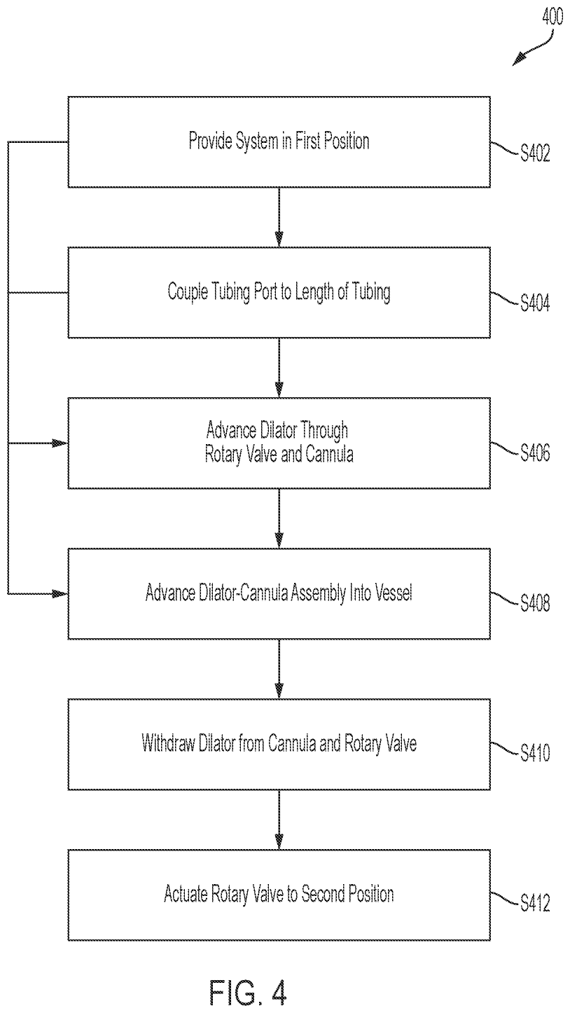

Referring now to FIG. 4, another aspect of the invention provides a method 400 of cannulating a vessel. The steps can be performed in a variety of orders. For example, the tubing can be coupled to the tubing port at a variety of points, if necessary.

In step S402, a system as described herein is provided in a first position so that the cannulated port and the dilator port are coupled.

In step S404, the tubing port can be coupled to a length of tubing.

In step S406, if not previously positioned, the dilator is advanced through the dilator distally through the rotary valve and the cannula.

In step S408, the dilator and the cannula are advanced into a vessel. For example, the dilator-cannula assembly can pierce a sidewall of a vessel. The vessel can be a blood vessel such as an umbilical blood vessel, e.g., an umbilical blood vessel of a pre-mature fetus.

In step S410, the dilator is withdrawn from the cannula and the rotary valve. In embodiments with a resiliently sealed dilator port, the dilator port is automatically sealed as the dilator is withdrawn.

In step S412, the valve is placed in a second position. This fluidically couples the cannulated vessel and the tubing.

The method can be performed by a single person. For example, a user can hold the rotary valve in a first hand and use a second hand to rotate the rotary valve to the first position, advance to the dilator, hold the vessel while advancing the dilator-cannula assembly into the vessel with the first hand, withdraw the vessel and rotate the rotary valve to the second position.

The method can be performed with less than 5 ml of blood loss and no air introduction.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A system comprising:
a cannula; and
a valve comprising:
a first port coupled to the cannula;
a second port adapted and configured for coupling to tubing;
a third port comprising a resilient pierceable seal; and
a central cylindrical plug received between the first port, the second port, and the third port, the central plug comprising a through-channel adapted and configured to selectively couple two of the ports;
wherein:
in a first position, the first port, the third port, and the through-channel are coupled such that a dilator can pass:
from outside the rotary valve;
through the pierceable seal;
through the first port; and
out a distal end of the cannula; and
in a second position, the first port and the second are port are fluidically coupled.

2. The system of claim 1, wherein the valve is a rotary valve.

3. The system of claim 2, wherein the second port and the third port can be rotated relative to the first port.

4. The system of claim 3, wherein the central cylindrical plug is fixed relative to the first port.

5. The system of claim 3, wherein the central cylindrical plug is fixed relative to the second port and the third port.

6. The system of claim 3, wherein the first port and the third port are axially aligned and/or diametrically opposed when in the first position.

7. The system of claim 2, wherein the first port and the third port are axially aligned or diametrically opposed.

8. The system of claim 1, wherein the dilator comprises a tapered distal end and a length relative to the cannula and the valve such that when the dilator is distally advanced through the rotary valve, the tapered distal end extends beyond a distal end of the cannula.

9. The system of claim 1, wherein the dilator is adapted and configured to facilitate puncturing of a blood vessel.

10. The system of claim 1, further comprising:
one or more centrally biased clamps adapted and configured to:
spread radially during puncture of a blood vessel; and
clamp the blood vessel against the cannula after puncture.

11. The system of claim 1, wherein the cannula has an outer diameter between about 7 French and about 16 French.

12. The system of claim 1, wherein in a third position, the through-channel is in fluidic communication with one or fewer of the ports.

13. The system of claim 1, wherein the valve is a piston valve.

14. A method of cannulating a vessel wherein fluid flows therethrough, the method comprising:
providing a valve comprising a first port, second port, and third port wherein a first port is coupled to a cannula and a second port is coupled to a length of tubing having a through channel;
introducing a dilator distally through the third port of the valve;
advancing the dilator and the cannula into the vessel;
withdrawing the dilator from the cannula and the valve; and
placing the valve in the second position to have the fluid flow between the vessel and the tubing.

15. The method of claim 14, wherein the vessel is a blood vessel.

16. The method of claim 15, wherein the blood vessel is an umbilical blood vessel.

17. The method of claim 14, wherein the method is performed by a single person.
